(19) United States
(12) Patent Application Publication
(10) Pub. No.: US 2017/0165395 A1
(43) Pub. Date: Jun. 15, 2017

(54) SYSTEMS FOR MANAGING INTIMATE SKIN

(71) Applicant: The Procter & Gamble Company, Cincinnati, OH (US)

(72) Inventors: Timothy Alan SCAVONE, Loveland, OH (US); Dean Larry DuVAL, Lebanon, OH (US); Nery Vanesa BRESLIN, Hamilton, OH (US)

(21) Appl. No.: 15/181,870

(22) Filed: Jun. 14, 2016

Related U.S. Application Data
(60) Provisional application No. 62/267,496, filed on Dec. 15, 2015.

(51) Int. Cl. A61L 15/44; A61K 8/67; A61Q 19/00; A61Q 9/02; A61K 8/02; A61K 8/92; A61K 8/58; A61K 8/891; A61Q 15/00; A61K 8/28; A61Q 13/00; A61K 8/11; A61K 8/81; A61K 8/31; A61L 15/46; B26B 21/40; A61M 35/00; A61F 13/551; B65D 69/00; A61K 8/34

(52) U.S. Cl. CPC A61L 15/44; A61K 8/345; A61K 8/673; A61Q 19/005; A61Q 9/02; A61K 8/0229; A61K 8/0216; A61K 8/92; A61K 8/585; A61K 8/891; A61Q 15/00; A61K 8/28; A61Q 13/00; A61K 8/11; A61K 8/8147; A61K 8/31; A61L 15/46; B26B 21/40; A61M 35/00; A61F 13/5519; B65D 69/00; A61K 2800/87; A61K 2800/31; A61L 2300/21; A61L 2300/216; A61K 2800/56; A61K 2800/592; A61K 2800/48

(57) ABSTRACT

Systems for managing intimate skin are described. The systems include a cosmetic product that comprises an applicator and a cosmetic composition disposed within the applicator. The cosmetic composition is solid at room temperature, such that some may describe the cosmetic composition as being in a stick form.

### FIELD OF THE INVENTION

The present invention relates to systems for managing intimate skin. Some of the systems can be used by groomers who shave or otherwise remove some or all of the hair growing on intimate skin. And some of the systems can be used by incontinent individuals who wear absorbent articles against intimate skin for relatively long periods of time.

### BACKGROUND OF THE INVENTION

Despite the large number of skin care products available in the market, there is still a need for a solution to everyday intimate hygiene and skin care especially for women. Women have special needs in the intimate area including the need for discreet intimate odor protection against vaginal odor and odor from sporadic unpredictable vaginal discharges that is particularly effective when these discharges occur. Moreover, an increasing trend toward intimate shaving has increased the demand for preventing irritation from hair removal, shave bumps following hair removal and preventing intimate itch as hair grows back following removal, as well as the need to facilitate healing of small cuts in the skin from intimate shaving. Additionally, many women facing problems with small urine and vaginal discharge leakage also need protection from skin rash that occurs from wearing an absorbent article all day that rubs against bare, hyper-hydrated skin.

Embodiments described herein are aimed at addressing the above problems.

### DETAILED DESCRIPTION OF THE INVENTION

The following text sets forth a broad description of numerous different embodiments of the present invention. The description is to be construed as exemplary only and does not describe every possible embodiment since describing every possible embodiment would be impractical, if not impossible. And it will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims. All publications and patents cited herein are incorporated herein by reference.

It should also be understood that, unless a term is expressly defined in this specification using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). No term is intended to be essential to the present invention unless so stated. To the extent that any term recited in the claims at the end of this patent is referred to in this patent in a manner consistent with a single meaning, that is done for sake of clarity only so as to not confuse the reader, and it is not intended that such a claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

The term “intimate skin” as used herein means the skin in the area between the waist and the upper thigh (e.g., the pelvic and pubis region). The compositions of the present invention can be employed for management of hair and skin associated with the pubis region.

The present invention relates to systems for managing intimate skin. The systems include a cosmetic product that comprises an applicator and a cosmetic composition disposed within the applicator. The cosmetic composition is solid at room temperature, such that some may describe the cosmetic composition as being in a stick form. The volume of the cosmetic composition may be appropriate for unit dose application wherein the applicator is disposed of after a single use. Alternatively, the volume of the cosmetic composition is appropriate for multiple, separate applications to intimate skin over the course of days and/or months.

Cosmetic compositions herein typically include a solvent (volatile and/or nonvolatile), a thickening agent, and one or more cosmetic ingredients. These ingredients are described in greater detail below.

The cosmetic composition of the present invention can comprise from about 10% to about 95%, preferably from about 20% to about 80%, more preferably from about 30% to about 70%, by weight of a volatile solvent suitable for topical application. The solvent being a major constituent of a composition which as a whole is required to be anhydrous is preferably anhydrous. “Volatile solvent” as used herein, refers to those materials that have measurable vapor pressure under ambient conditions. The volatile solvent can comprise a volatile silicone liquid. The concentration of the volatile silicone ranges from about 10% to about 90%, more preferably from about 15% to about 65%, even more preferably from about 30% to about 60%, by weight of the cosmetic stick composition. The volatile silicone may be a cyclic, linear or branched chain silicone having the requisite volatility as defined herein. Non-limiting examples of suitable volatile silicones are described in Todd et al., “Volatile Silicone Fluids for Cosmetics”, Cosmetics and Toiletries, 91:27-32 (1976), which descriptions are incorporated herein by reference. Preferred among these volatile silicones are the cyclic silicones having from about 3 to about 7, more preferably from about 5 to about 6, silicon atoms. Most preferably are those which conform to the formula:

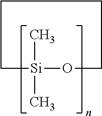

wherein n is from about 3 to about 7, preferably from about 5 to about 6, most preferably 5. These volatile cyclic silicones generally have a viscosity value of less than about 10 centistokes. All viscosity values described herein are measured or determined under ambient conditions, unless otherwise specified. Examples of suitable volatile silicones for use herein include Cyclomethicone D-5 (commercially available from G. E. Silicones); Dow Corning 344, and Dow Corning 345 (commercially available from Dow Corning Corp.); and GE 7207, GE 7158 and Silicone Fluids SF-1202 and SF-1173 (available from General Electric Co.).

In addition to volatile silicone liquids the volatile solvent can also comprise volatile, nonpolar hydrocarbon liquids. In this context, the term “nonpolar” means that these volatile hydrocarbon liquids have a solubility parameter of less than about 7.5 (cal/cm³)^{0.5}, most typically about 5.0 (cal/cm³)^{0.5} to less than about 7.5 (cal/cm³)^{0.5}. These volatile, nonpolar hydrocarbon liquids preferably contain only hydrogen and carbon and therefore preferably contain no functional groups. Solubility parameters as described above are determined by methods well known in the chemical arts for establishing the relative polar character of a solvent or other material. A description of solubility parameters and means for determining them are described by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988, which descriptions are incorporated herein by reference.

The nonpolar, volatile hydrocarbon liquid as a liquid carrier for use in the composition of the present invention is preferably a liquid paraffin and/or isoparaffin having the requisite volatility and nonpolar character. The nonpolar, volatile hydrocarbon liquids can have a cyclic, branched and/or chain configuration, and can be saturated or unsaturated, preferably saturated.

Preferred volatile, nonpolar hydrocarbon liquids are branched chain hydrocarbons at a concentration of from about 1% to about 40%, more preferably from about 1% to about 20%, by weight of the composition, and having from about 6 to about 40 carbon atoms, preferably from about 6 to about 20 carbon atoms. These preferred hydrocarbon liquids will most typically be formulated as a combination of two or more of the above-described branched chain hydrocarbons, wherein the combination of two or more hydrocarbons have different molecular weights, number of carbon atoms, and/or chain configurations. Specific nonlimiting examples of such combinations include the isoparaffins available from Exxon Chemical Company, Baytown, Tex. U.S.A, sold as Isopar M (C13-C14 Isoparaffin), Isopar C (C7-C8 Isoparaffin), Isopar E (C8-C9 Isoparaffin), Isopar G (C10-11 Isoparaffin), Isopar L (C11-C13 Isoparaffin), Isopar H (C11-C12 Isoparaffin), and combinations thereof. Other nonlimiting examples of suitable branched chain hydrocarbons include Permethyl 99A (C12, isododecane), Permethyl 101A (C16, isohexadecane), Permethyl 102A (C20, isoeicosane), and combinations thereof. The Permethyl series are available from Presperse, Inc., South Plainfield, N.J., U.S.A. Other nonlimiting examples of suitable branched chain hydrocarbons include petroleum distallates such as those available from Phillips Chemical as Soltrol 130, Soltrol 150, Soltrol 170, and those available from Shell as Shell Sol-70, -71, and -2033.

Still other suitable isoparaffins include C9-C11 Isoparaffin, C9-C13 Isoparaffin, C9-C14 Isoparaffin, C10-C13 Isoparaffin, C12-C14 Isoparaffin, C13-C16 Isoparaffin, C14-C18 Isoparaffin, and hydrogenated polyisobutene available from Amoco as the Panalane Series and from Fanning Corporation as the Fancor P series.

Nonlimiting examples of other volatile, nonpolar hydrocarbon liquids suitable for use in the cosmetic compositions include paraffins such as dodecane, octane, decane and combinations thereof, and the Norpar series of paraffins available from Exxon Chemical Company such as Norpar-12, -13, and -15 and the Neosolve series of paraffins available from Shell. Yet another example includes C11-C15 alkanes/cycloalkanes, such as those available from Exxon as Exxsol D80.

The cosmetic compositions can also comprise a non-volatile solvent. These non-volatile solvents may be either non-volatile organic fluids or non-volatile silicone fluids. The non-volatile organic fluid can be present at concentrations ranging from about 1%, from about 2% but no more than about 20% or no more than about 15%, by weight of the composition. Non-limiting examples of nonvolatile organic fluids include, but are not limited to, mineral oil, PPG-14 butyl ether, isopropyl myristate, petrolatum, butyl stearate, cetyl octanoate, butyl myristate, myristyl myristate, C12-15 alkylbenzoate (e.g., Finsolv™)), dipropylene glycol dibenzoate, PPG-15 stearyl ether benzoate and blends thereof (e.g. Finsolv TPP), neopentyl glycol diheptanoate (e.g. Lexfeel 7 supplied by Inolex), octyldodecanol, isostearyl isostearate, octododecyl benzoate, isostearyl lactate, isostearyl palmitate, isononyl/isononoate, isoeicosane, octyldodecyl neopentanate, hydrogenated polyisobutane, and isobutyl stearate. Many such other carrier liquids are disclosed in U.S. Pat. No. 6,013,248 (Luebbe et al.) and U.S. Pat. No. 5,968,489 (Swaile et al).

The non-volatile silicone fluid may be a liquid at or below human skin temperature, or otherwise in liquid form within the composition during or shortly after topical application: The concentration of the non-volatile silicone may be from about 1%, from about 2% but no more than about 15% or no more than about 10%, by weight of the composition. Nonvolatile silicone fluids of the present invention may include those that conform to the formula:

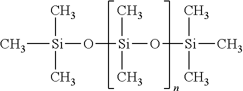

wherein n is greater than or equal to 1. These linear silicone materials may generally have viscosity values of from about 5 centistokes or from about 10 centistokes to about 100,000 centistokes, or to about 500 centistokes or to about 200 centistokes or to about 50 centistokes, as measured under ambient conditions. Specific non limiting examples of suitable nonvolatile silicone fluids include Dow Corning 200, hexamethyldisiloxane, Dow Corning 225, Dow Corning 1732, Dow Corning 5732, Dow Corning 5750 (available from Dow Corning Corp.); and SF-96, SF-1066 and SF 18(350) Silicone Fluids (available from G.E. Silicones).

Low surface tension non-volatile silicone fluids may be also be used. Such solvents may be selected from the group consisting of dimethicones, dimethicone copolyols, phenyl trimethicones, alkyl dimethicones, alkyl methicones, and mixtures thereof. Low surface tension non-volatile solvents are also described in U.S. Pat. No. 6,835,373 (Kolodzik et al.).

In general it is preferred that in the cosmetic composition the weight ratio of volatile to non-volatile solvent is at least 1:1 and more preferably at least 2:1. Using these preferred ratios provides the additional benefit of reducing the risk of staining the garments which come in contact with the skin treated with the compositions of the invention.

The cosmetic composition compositions generally include a thickening agent to help provide the composition with the desired viscosity, rheology, texture and/or product hardness, or to otherwise help suspend any dispersed solids or liquids within the composition. The term “thickening agent” may include any material known or otherwise effective in providing suspending, gelling, viscosifying, solidifying or thickening properties to the composition or which otherwise provide structure to the final product form. These thickening agents may include gelling agents, polymeric or nonpolymeric agents, inorganic thickening agents, or viscosifying agents. The thickening agents may include organic solids, silicone solids, crystalline or other gellants, inorganic particulates such as clays or silicas, or combinations thereof. Preferred thickening agents are gellants, for example triglyceride gellants.

The concentration and type of the thickening agent selected for use in the cosmetic compositions will vary depending upon the desired product form, viscosity, and hardness. The thickening agents suitable for use herein, may have a concentration range from 2% to 45% by weight of the composition.

Non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof. Concentrations of all such gelling agents may be from at least about 0.1%, at least about 1%, or at least about 5% and no more than about 25%, no more than about 15%, or no more than about 10%, by weight of the composition.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

Other suitable gelling agents include amide gellants such as disubstituted or branched monoamide gellants, monosubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable amide gelling agents are described in U.S. Pat. No. 5,429,816, issued Jul. 4, 1995, and U.S. Pat. No. 5,840,287, filed Dec. 20, 1996.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C(Syncrowax® available from Croda, Inc.).

Other suitable thickening agents include waxes or wax-like materials having a melt point of above 65° C., more typically from about 65° C. to about 130° C., examples of which include, but are not limited to, waxes such as beeswax, carnauba, bayberry, candelilla, montan, ozokerite, ceresin, hydrogenated castor oil (castor wax), synthetic waxes and microcrystalline waxes. Castor wax is preferred within this group. Other high melting point waxes are described in U.S. Pat. No. 4,049,792, Elsnau, issued Sep. 20, 1977.

Further thickening agents for use in the cosmetic compositions include inorganic particulate thickening agents such as clays and colloidal pyrogenic silica pigments. For example, colloidal pyrogenic silica pigments such as Cab-O-Sil®, a submicroscopic particulated pyrogenic silica may be used. Other known or otherwise effective inorganic particulate thickening agents that are commonly used in the art can also be used in the solid antiperspirant compositions of the present invention. Concentrations of particulate thickening agents may range, for example, from at least about 0.1%, at least about 1%, at least about 5% but no more than about 35%, no more than about 15%, no more than about 10% or no more than about 8%, by weight of the composition.

Suitable clay thickening agents include montmorillonite clays, examples of which include bentonites, hectorites, and colloidal magnesium aluminum silicates. These and other suitable clays may be hydrophobically treated, and when so treated will generally be used in combination with a clay activator. Non-limiting examples of suitable clay activators include propylene carbonate, ethanol, and combinations thereof. When clay activators are present, the amount of clay activator will typically range from at least about 40%, at least about 25%, at least about 15% but no more than about 75%, no more than about 60%, or no more than about 50%, by weight of the clay.

The cosmetic compositions are preferably in the form of a solid composition, e.g., as a solid stick. When the stick is rubbed onto the skin a thin layer of composition is applied onto the skin due to the mechanical action and/or the temperature of the body which partially melts the solid stick material on its surface. Solid stick product forms can hold an advantage over other product forms (such as, for example, lotions, creams and gels) because no drying or waiting is required before donning one's undergarments after rubbing the composition onto skin/hair in the intimate area. Solid stick product forms can also hold an advantage over sprays and other non-touch applications in that the user can more easily and accurately control application of the composition to skin the intimate area. Besides not requiring drying after application of a solid stick composition, but use of this form can provide a dryer feeling post application and while wearing an absorbent article. Tables 1 and 2 below illustrate this dry feeling in comparison to compositions in a lotion or spray form.

TABLE 1
Consumer Use Test (Rating 1-5, higher being better)

| | Lotion and Spray | Spray | Absorbent |
|---|---|---|---|
| | Application | application | article |
| | followed by wearing | followed by wearing | usage |
| Attribute | an absorbent article | an absorbent article | alone |
| Dry during wear | 3.70 | 3.71 | 3.55 |

TABLE 2
Consumer Use Test (Rating 1-5, higher being better)

| | Solid stick | Absorbent article |
|---|---|---|
| | application | alone or with |
| | followed by wearing | consumer's own |
| Attribute | an absorbent article | cosmetic product |
| Leaves my skin feeling dry | 4.13 | 2.75* |

*statistically significant at 95% CI

As one can see from Table 1, use of a lotion or spray cosmetic product form did not change the perceived feeling of dryness, while Table 2 illustrates use of a solid stick provided a dryer skin feeling while wearing an absorbent article.

Solid stick compositions may comprise 5-45% wt. of a thickening agent which is a gellant and have a product hardness of at least about 600 gram.force, most typically from about 600 gram.force to about 5,000 gram.force, preferably from about 750 gram.force to about 2,000 gram.force, more preferably from about 800 gram.force to about 1,400 gram.force. The term “product hardness” or “hardness” as used herein is a reflection of how much force is required to move a penetration cone a specified distance and at a controlled rate into a cosmetic stick composition under the following test conditions. Higher values represent harder product, and lower values represent softer product. These values are measured at 27° C., 15% relative humidity, using a TA-XT2 Texture Analyzer, available from Texture Technology Corp., Scarsdale, N.Y., U.S.A. The product hardness value as used herein represents the peak force required to move a standard 45 DEG angle penetration cone through the composition for a distance of 10 mm at a rate of 2 mm/second. The standard cone is available from Texture Technology Corp., as part number TA-15, and has a total cone length of about 24.7 mm, angled cone length of about 18.3 mm, a maximum diameter of the angled surface of the cone of about 15.5 mm. The cone is a smooth, stainless steel construction and weighs about 17.8 grams.

The cosmetic compositions of the present invention may contain one or more cosmetic active ingredients. When present, the cosmetic actives will be present at a level of from about 0.01% to about 60% by weight of the cosmetic composition. Suitable cosmetic actives include any known or otherwise effective cosmetic active that is compatible with the essential ingredients of the cosmetic sticks of the present invention, or which do not otherwise unduly impair the product performance thereof. Cosmetic actives suitable for use in the compositions of the present invention include moisturizers, emollients, perfumes or fragrances, skin conditioners, antioxidants, vitamins, anti-wrinkle products, anti-itch products (e.g., antihistamines such as diphenhydramine, corticoid steroids such as hydrocortisone and anesthetics such as benzocaine), surfactants, pharmaceuticals, deodorants, colorants, pigments, sunscreens or other photo protectants, topical hormones such as estrogens, isoflavones such as genistein and daidzein, materials which provide a tactile sensation of heat or cold such as menthyl lactate, and any other inert or active material intended or otherwise suitable for topical application to the skin.

A representative, non-limiting list of cooling agents includes menthol, menthone, isopulegol, N-ethyl p-menthanecarboxamide (WS-3), N,2,3-trimethyl-2-isopropylbutanamide (WS-23), menthyl lactate (Frescolat® ML), menthone glycerine acetal (Frescolat® MGA), mono-menthyl succinate (Physcool®), mono-menthyl glutarate, O-menthyl glycerine (CoolAct® 10), menthyl-N,N-dimethylsuccinamate or 2-sec-butylcyclohexanone (Freskomenthe®). Such conventional cooling materials tend to have an enthalpy of vaporization of less than 71 kJ/mol at 760 Torr, and preferably less than 65 kJ/mol at 760 Torr. For example, N-ethyl p-menthanecarboxamide (WS-3), N,2,3-trimethyl-2-isopropylbutanamide (WS-23), menthol, menthyl lactate, ethyl 3-(p-menthane-3-carboxamide) acetate (WS-5), menthone glycerine acetal, menthoxypropane-1,2-diol, isopulegol, p-menthane-3,8-diols, N,N-dimethyl menthyl succinamide, (3S,3aR,3bR,4S,7R,7aR)-Octahydro-3,7-dimethyl-4-(1-methylethyl)-1H-cyclopenta[1,3]cyclopropa[1,2]benzen-3-ol (Cubebol®), and menthyl PCA, have enthalpy of vaporization values of approximately 58.4 kJ/mol at 760 Torr, approximately 47 kJ/mol at 760 Torr, approximately 52.5 kJ/mol at 760 Torr, approximately 63.1 kJ/mol at 760 Torr, approximately 64 kJ/mol at 760 Torr, approximately 65.5 kJ/mol at 760 Torr, approximately 70.5 kJ/mol at 760 Torr, approximately 50.4 kJ/mol at 760 Torr, approximately 58.7 kJ/mol at 760 Torr, approximately 63.4 kJ/mol at 760 Torr, approximately 60.2 kJ/mol at 760 Torr, and approximately 66.1 kJ/mol at 760 Torr, respectively.

Specific examples of cosmetic actives suitable for use herein include deodorant actives, perfumes and fragrances, antimicrobials (antibacterial, antifungal), steroidal anti-inflammatory materials (e.g., hydrocortisone), non-steroidal anti-inflammatory materials, vitamins and derivatives thereof (e.g., thiamin, riboflavin, niacin, pyridoxine, vitamin A, vitamin D, vitamin E, vitamin K), hydroxy and alpha-hydroxy acids (e.g., salicylic acid, citric acid), moisturizers (e.g., silicone and non-silicone), and the like.

Particularly preferred cosmetic actives for use herein are Zinc Oxide, Petrolatum, dimethicone, Panthenyl Triacetate, Lactic Acid, Vitamin B₃ and derivatives, Vitamin B₅ and derivatives, green tea extract, hexamidine, polycarbophil, pectin, and mixtures thereof. Derivatives of Vitamin B₅ are believed to improve the condition of skin and to accelerate the rate of wound healing. This can be particularly advantageous when applied to intimate area skin that is irritated or cut during shaving. Liquid derivatives of Vitamin B₅ are also believed to soften hair. Over repeated uses, this can allow a consumer to experience an easier or closer shave, and can make the pubic hair less itchy when it grows back. Lastly, it is believed that derivatives of Vitamin B₅ can reduce the number and extent of shave bumps that occur from ingrown hair. These advantages appear to be more noticeable in the intimate area where sensitivity to itch, irritation, shave bumps, and hair regrowth is greater. Benefits can be noticeable in 1-2 days versus one week in the underarm. To be able to formulate Vitamin B₅ into anhydrous media it is helpful to derivatize the vitamin so that it is sufficiently soluble in the anhydrous carrier while still retaining bioavailability for would healing properties. In one approach the degree of substitution may be 1.5-3 where the substitution chemistry contains one or more carbons and the derivative remain liquid at 75° F. (hair temperature) so that it can effectively plasticize and soften the hair shaft.

The cosmetic compositions may optionally contain one or more compounds for managing odors, including reactive compounds, encapsulated reactive compounds, complexed compounds, cosmetic actives, and formulation raw materials. These ingredients are described in greater detail below.

Examples of reactive compounds suitable for use in the present invention are: melonal, adoxal, trans-2-hexenal, ligustral, Floral Super, Florhydral, 5-methyl-2-thiophene-carboxaldehyde, hydratropic aldehyde, undecenal, 9-undecenal, 10-undecenal, trans-4-decenal, cis-6-nonenal, isocyclocitral, precyclemone b, (E)-2-(z)-6-nonadienal, undecyl aldehyde, methyl-octyl-acetaldehyde, Lauric aldehyde, silvial, vanillin, floralozone.

The reactive compounds can optionally be encapsulated using any technique known in the art. The term “encapsulation” within the present invention is intended to encompass any technology which allows introducing a reactive compound according to the invention into a composition according to the invention in a mixture with other materials which are called in general “encapsulating materials”. The reactive compounds when encapsulated are inhibited from contacting other materials so to avoid unwanted reactions. Moreover, when encapsulated, their evaporation is inhibited. Many types of capsules are known in the art and are used for the delivery of perfume ingredients. Capsules can have any size, typically used in the art and suitable herein are nanocapsules, microcapsules, and larger capsules. Capsules allow the encapsulated composition to release when it is needed. Typically in the case of the present invention this corresponds to two cases: one, when the skin receives a liquid insult (e.g. when urine, menses, sweat or vaginal fluids are discharged): in this case capsules comprise water soluble materials or materials which trigger release of the encapsulated compound when contacted with water or a water containing liquid; and two, when pressure or force is exerted on the capsules, e.g., during application of the topical composition or later once the composition is applied onto the skin due to normal movement, abrasion between neighboring sections of skin or between the underwear and the skin. In this case, for example, breakable capsules having a shell of rupturable polymeric film can be used. Other trigger (or combination of triggers) mechanisms can be used to release the encapsulated compound from the capsule, e.g. evaporation, diffusion, temperature, humidity, light etc. The release of the encapsulated compound can be instantaneous or sustained over time, depending on needs. The skilled person, based on the desired trigger action and release type, will be able to select the appropriate encapsulating material from those known in the art.

Polymeric materials can be used as encapsulating materials. Classical coacervates, water soluble or partly soluble to insoluble charged or neutral polymers, liquid crystals, hot melts, hydrogels, perfumed plastics, microcapsules, nano- and micro-latexes, polymeric film formers, and polymeric absorbents, polymeric adsorbents, etc. are some examples.

The use of a starch encapsulation technology allows one to modify the properties of the compound to be encapsulated, for example, by converting a liquid compound into a solid by adding ingredients such as starch. The benefit includes increased retention for volatile compounds during product storage. Upon exposure to moisture, a release may be triggered. Suitable starches can be made from raw starch, pregelatinized starch, modified starch derived from tubers, legumes, cereal and grains for example corn starch, wheat starch, rice starch, waxy corn starch, oat starch, cassava starch, waxy barley starch, waxy rice starch, sweet rice starch, amioca, potato starch, tapioca starch and mixtures thereof. Modified starches may be particularly suitable and they can include hydrolyzed starch, acid thinned starch, starch having hydrophobic groups, such as starch esters of long chain hydrocarbons (C₅ or greater), starch acetates, starch octenyl succinate and mixtures thereof. In one aspect, starch esters, such as starch octenyl succinates are employed. The term “hydrolyzed starch” refers to oligosaccharide-type materials that are typically obtained by acid and/or enzymatic hydrolysis of starches, preferably corn starch. It may be preferred to include in the starch water-mixture, a starch ester. Particularly preferred are the modified starches comprising a starch derivative containing a hydrophobic group or both a hydrophobic and a hydrophilic group which has been degraded by at least one enzyme capable of cleaving the 1,4 linkages of the starch molecule from the non-reducing ends to produce short chained saccharides to provide high oxidation resistance while maintaining substantially high molecular weight portions of the starch base. The aqueous starch mixture may also include a plasticizer for the starch. Suitable examples include monosaccharides, disaccharides, oligosaccharides and maltodextrins, such as glucose, sucrose, sorbitol, gum arabic, guar gums and maltodextrin.

The primary materials forming the capsule as described so far, may be further encapsulated with a secondary coating material. Any of the capsule types mentioned so far can be used in the present invention as such or with an additional secondary coating material. An additional secondary coating material can help in reducing the scent perception, in reducing evaporation of volatile components over time (especially at elevated temperatures and humidity conditions) and in increasing chemical stability of the complexed compound by reducing the exposure of the complexed compounds (which in the present invention comprise highly reactive materials) to prematurely react or decompose so they are no longer functional or have a different odor character when activated. Additionally the use of coated capsules can allow altering the release characteristic of the encapsulated material (slowing or accelerating its release, or changing the release trigger, for example introducing a pH trigger). Generally, any second material that is added to or applied directly to a primary encapsulating material that accomplishes one or more of the above functions is characterized as a coating. The secondary coating may be directly applied using a second process step following creation of the primary capsule, using a process such as prilling, or using any fluidized bed process to apply a secondary surface coating (for example a Wurster Coater).

Coating compositions can include, for example, polysaccharides (for example, but not limited to unmodified starch, chemically modified starch, dextrins, cyclodextrin and cyclodextrin derivatives), natural and artificial/synthetic waxes, esters and ester derivatives, fatty acids, natural and synthetic and chemically modified lipids, fatty alcohols, hydrocarbons (linear or branched, petrolatum), enteric coating compositions (such as the Eudragit series of Methacrylic acid co-polymers), polyvinyl alcohols, polyethylene glycols, silicones (for example, but not limited to silicone copolymers and functionalized silicones), surfactants, emulsifiers, polypropylene glycols, cellulose derivatives (methyl cellulose, hydroxypropyl cellulose), glycerin, mono and diglycerides, polyglycerol and polyglycerol esters and emulsifiers employed in food applications.

As noted above, the cosmetic composition may contain complexed compounds for managing odors. For “complex” it is intended an “inclusion complex” within the meaning of IUPAC Compendium of Chemical Terminology 2nd Edition (1997) wherein the complexing agent is the host and the complexed compound is the “guest”. Examples of complexing agents are cyclodextrins, which includes any of the known cyclodextrins such as substituted and unsubstituted cyclodextrins containing from about six to about twelve glucose units, for example alpha-cyclodextrin, beta-cyclodextrin, gamma-cyclodextrin and/or their derivatives and/or mixtures thereof. The cyclodextrin complex can comprise cyclodextrin selected from the group consisting of beta-cyclodextrin, alpha-cyclodextrin, hydroxypropyl alpha-cyclodextrin, hydroxypropyl beta-cyclodextrin, methylated-alpha-cyclodextrin, methylated-beta-cyclodextrin, and mixtures thereof.

The cosmetic compositions herein may comprise one or more complexed or encapsulated compounds having a “thiol vapor pressure suppression index” (TVPS) of more than 20. Examples of compounds that have a TVPS higher than 20 are those of the following list (a): melonal, adoxal, trans-2-hexenal, ligustral, Floral Super, Florhydral, 5-methyl-2-thiophene-carboxaldehyde, hydratropic aldehyde, undecenal, 9-undecenal, 10-undecenal, trans-4-decenal, cis-6-nonenal, isocyclocitral, precyclemone b, (E)-2,(z)-6-nonadienal, undecyl aldehyde, methyl-octyl-acetaldehyde, Lauric aldehyde, silvial, vanillin, floralozone. These compounds not only have a TVPS higher than 20 but also they form complexes and/or capsules which are particularly stable and release the complexed encapsulated materials when needed.

The compounds in list (a) are particularly reactive toward malodorant molecules containing Sulphur atoms (thiol type malodors, typically associated with protein degradation e.g. in menstrual fluids, feces, food etc). The primary function of the complexed or encapsulated reactive compounds is to chemically react with malodors, such as malodorant molecules containing Nitrogen atoms (amine type odors, typically deriving from the degradation of urine or certain foods like onions) and/or malodorant molecules containing Sulphur atoms (thiol type malodors, typically associated with protein degradation, e.g., in menstrual fluid). Ammonia/amines are one component of malodor associated with the absorption of bodily fluids, such as menses or urine. For example, ammonia/amines are typically present in high amounts in absorbent products used for urine absorption due to degradation of urea Ammonia/amines and their derivatives can react with aldehydes and/or ketones to form imines (according to the so-called Schiff base reaction).

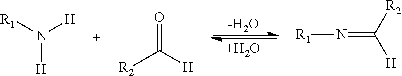

This reaction is catalyzed by enzymes and/or by a slightly acidic pH 4 to 5. The moderate acid requirement is necessary to allow protonation of the hydroxyl intermediate to allow water to leave. Malodorant sulphur based compounds are typically generated by the degradation of proteins e.g. in menstrual fluids and feces so their control is particularly important in an intimate hygiene context. The mechanism of action is not fully understood at the moment, but it is believed that it is connected to the fact that Thiols can react with aldehydes and ketones to form thioacetals and thioketals.

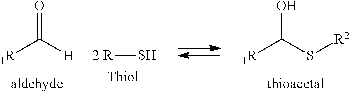

In principle, the chemical reactions described above can be obtained from any aldehyde, but in practice the reactivity of aldehydes in these type of reactions is very different. The reactive compounds described herein are effective in reacting with Nitrogen based malodorant molecules and particularly effective in reacting with sulphur based malodorant molecules. The particularly high reactivity of the reactive compounds of the invention towards sulphur based malodorant molecules renders the present invention particularly effective for use in the intimate area.

Other selected additional compounds in complexed or encapsulated form can be optionally used alternatively to or in combination with the reactive compounds having a TVPS of more than 20, described above in list (a). Suitable exemplary compounds are listed below in lists (b), (c), (d) and (e). Suitable selected aldehydes and/or ketones include: (b): hexyl cinnamic aldehyde, alpha-amylcinnamic aldehyde, p-anisaldehyde, benzaldehyde, cinnamic aldehyde, cuminic aldehyde, decanal, cyclamen aldehyde, p-t-butyl-alpha-methyldihydrocinnamaldehyde, 4-hydroxy-3-methoxycinnamaldehyde, vanillin isobutyrate, 2-phenyl-3-(2-furyl)prop-2-enal, ethyl vanillin acetate, vanillin acetate, heptanal, lauryl aldehyde, nonanal, octanal, phenylacetaldehyde, phenyl propyl aldehyde, salycil aldehyde, citral, 2,4-dihydroxy-3-methylbenzaldehyde, 2-hydroxy-4-methylbenzaldehyde, 5-methyl salicylic aldehydes, 4-nitrobenzaldehyde, o-nitrobenzaldehyde, 5-ethyl-2-thiophenecarbaldehyde, 2-thiophenecarbaldehyde, asaronaldehyde, 5-(hydroxymethyl)-2-furaldehyde, 2-benzofurancarboxaldehyde, 2,3,4-trimethoxybenzaldehyde, protocatechualdehyde, heliotropine, 4-ethoxy-3-methoxy benzaldehyde, 3,4,5-trimethoxybenzaldehyde, 3-hydroxybenzaldehyde, o-methoxycinnamaldehyde, 3,5-dimethoxy-4-hydroxycinnamaldehyde, 2,8-dithianon-4-3n-4-carboxaldehyde, sorbinaldehyde, 2,4-heptadienal, 2,4-decadienal, 2,4-nonadienal, 2,4-nonadienal, (E,E)-,2,4-octadien-1-al, 2,4-octadienal, 2,4-dodecadienal, 2,4-undecadienal, 2,4-tridecadien-1-al, 2-trans-4-cis-7-cis-tridecatrienal, piperonylidene propionaldehyde, 2-methyl-3-(2-furyl)acrolein, 2,4-pentadienal, 2-furfurylidene butyrraldehyde, helional, lyral, 3-hexenal, safranal, veratraldehyde, 3-(2-furyl)acrolein, pyruvaldehyde, ethanedial, 1-(2,6,6-trimethyl-1-cyclohexenyl)pent-1-en-3-one; 4-(2,6,6-trimethyl-1-cyclohexen-1-yl)-3-Buten-2-one; 4-(2,6,6-trimethyl-2-cyclohexen-1-yl)-3-buten-2-one, 5-(2,6,6-Trimethyl-2-cyclohexen-1-yl)4-penten-3-one, (E)-4-(2,2-dimethyl-6-methylidenecyclohexyl)but-3-en-2-one.

Components from list (c) are menthol, menthyl acetate, menthyl lactate, menthyl propionate, menthyl butyrrate, menthone, mint terpenes, laevo-carvone, Cis-3-Hexenol & Cis-3-Hexenyl acetate, koavone and methyl dioxolan.

These are all compounds whose primary function is to mask malodors. This may occur through vapor pressure suppression of the malodor or by overwhelming the unpleasant malodor with the pleasant odor of the fragrance component. These materials, when used, may significantly reduce the ability to detect the malodors. The masking ability to hide malodors is possible due to the volatile nature of the materials selected, which are released from the complex or capsule in the composition for topical use and are then inhaled into the nose of a user, generally within somewhat close range, e.g., within about 0 to 10 meters of the person by normal breathing (although this should in no way be intended to limit the scope of the invention).

Components from class (d) are methyl-dihydrojasmonate, methyl jasmonate, eucalyptol, tetrahydro-linalool, Phenyl-Ethyl alcohol, Hexyl iso-butyrate, Linalyl acetate, Benzyl acetate, Benzyl alcohol, or mixtures thereof. These are volatile materials which are well complexed in particular when the complexing agent is a cyclodextrin and are release very quickly upon contact with a water based liquid. Their presence allows the topical composition to respond more quickly to an insult of malodorant substances by releasing compounds that have a good general masking effect against malodors, in particular, being very volatile, reduces the vapor pressure of other malodorant compounds slowing down their evaporation rate.

Other suitable malodor masking and fragrance components which can optionally be employed include those in the following list (e): camphor, p-menthane, limonene, cresol, linalool, myrcenol, tetra hydromyrcenol, di-hydromyrcenol, myrcene, citronellol, citronellyil derivatives, geraniol, geranyl derivatives, mugetanol, eugenol, jasmal, terpineol, pinanol, cedrene, damascone, beta pinene, cineole and its derivatives, nonadienol, ethylhexanal, octanol acetate, methyl furfural, terpinene, thujene, amylacetate, camphene, citronellal, hydroxycitronellal, ethyl maltol, methyl phenyl carbinyl acetate, dihydrocumarin, di-hydromyrcenyl acetate, geraniol, geranial, isoamylacetate, ethyl, and/or triethyl acetate, para-cresol, para-cymene, methyl abietate, hexyl-2-methyl butyrate, hexyl-2-methyl butyrate, and mixtures thereof.

All the compounds mentioned herein, unless a specific isomeric form is specified, also include their isomeric forms, diastereomers and enantiomers. In addition to the components from lists (a), (b), (c), (d) and (e) in a complexed or encapsulated form, the cosmetic compositions can include components from the same lists or other fragrance components in free form (i.e., not complexed or encapsulated).

In some embodiments, the cosmetic compositions are anhydrous meaning they contain less than about 2%, 1%, 0.5%, or 0% of added water that is a separate ingredient and that is not associated with other compositional ingredients. The cosmetic compositions can also be limited in glycol content (e.g., propylene glycol and dipropylene glycol), meaning the compositions contain less than about 1% of a glycol.

Tables 3 and 4 include non-limiting examples to illustrate specific embodiments of the cosmetic stick compositions of the present invention. Each is formulated by combining the solid thickening agents (gellants) and liquid carriers in a vessel equipped with a heat source. The combined solids and liquids are heated to a temperature ranging from 85° C. to 96° C. and agitated to dissolve the solid thickening agents until the mixture forms a homogeneous clear to slightly cloudy solution, at which point any solid cosmetic active is added to and dispersed throughout the heated solution while maintaining mixing. The complexed or encapsulated compounds are then added to the melted mixture. If using a cyclodextrin complex it is preferable to use a cyclodextrin complex with a degree of complexing greater than about 75% to prevent perfume from flashing or evaporating in the heating stage of the making process and to prevent compounds from the complex from blending in with the free perfume added separately. If needed, the mixture may then be milled to eliminate solid particles or clumps and render the composition smooth and free of large particles. It is especially preferred to use a spray dried cyclodextrin complex because the particles are free of hard glassy edges that will scratch the skin during use. Other methods of producing the complex (for example via extrusion, or via solvent crystallization) create hard glassy particles that scratch the skin. Finally, an optional perfume may be added at this stage, just prior to or after cooling. The resulting heated combination is then circulated through a scraped wall heat exchanger and cooled to 62° C. before filling the cooled mixture into plastic dispensing canisters and allowed to cool and solidify within the canisters over a 20 minute period (cooling rate of 2° C./min) through a forced air cooling tunnel having an air temperature of 21° C. The exemplified compositions are then placed in a constant temperature room maintained at 25° C. for a period of one week (7 days) after which they are evaluated for hardness according to the method described herein. Each of the exemplified compositions is applied topically to the appropriate area of the skin, in accordance with the methods of use described herein. All exemplified amounts are weight percentages based upon the total weight of the cosmetic stick composition, unless otherwise specified.

TABLE 3
Exemplary Cosmetic Compositions

| Ingredient | Example 1 | Example 2 |
|---|---|---|
| Ozokerite Wax | 16.00 | 17.00 |
| Cyclopentasiloxane | 46.81 | 46.81 |
| Tribehenin (Syncrowax HR-C)¹ | 0.00 | 0.00 |
| C18-36 Acid Triglyceride (Syncrowax HGLC)² | 0.00 | 0.00 |
| Perfume | 0.00 | 0.00 |
| Petrolatum | 4.00 | 5.00 |
| Spray Dried Beta Cyclodextrin Feminine | 5.00 | 5.00 |
| Odor Control Perfume Complex (95% Degree | | |
| Of Complexing) | | |
| Zinc Oxide | 5.00 | 0.00 |
| Dimethicone 50 cst | 3.00 | 3.00 |
| C12-C15 Alkyl Benzoate | 10.00 | 10.00 |
| PPG 14 Butyl Ether (Fluid AP) | 10.00 | 10.00 |
| Behenyl Alcohol | 0.19 | 0.19 |
| Panthenyl Triacetate | 0.00 | 3.00 |
| Spray Dried Polyacrylate Microcapsules | 0.00 | 0.00 |
| Containing Feminine Odor Control Perfume | | |
| Added Water | 0.00 | 0.00 |
| Antiperspirant Active (e.g., Aluminum | 0.00 | 0.00 |
| Zirconium Tetrachlorohydrex Gly) | | |
| Propylene Glycol/Dipropylene Glycol | 0.00 | 0.00 |
| | (1.00<) | (1.00<) |
| | (0.10<) | (0.10<) |
| Totals | 100.00 | 100.00 |
| Product hardness (gram · force) | 1102 | 920 |

TABLE 4
Exemplary Cosmetic Compositions

| Ingredient | Example 3 | Example 4 |
|---|---|---|
| Cyclomethicone | 45.99 | 48.99 |
| Fluid AP | 5.00 | 5.00 |
| Dimethicone 50 cst | 5.00 | 5.00 |
| Mineral Oil | 8.00 | 8.00 |
| Petrolatum | 5.00 | 5.00 |
| Stearyl Alcohol | 16.00 | 16.00 |
| Hydrogenated Castor Oil | 3.56 | 3.56 |
| Behenyl Alcohol | 0.20 | 0.20 |
| Performathox 450 Ethoxylate | 1.00 | 1.00 |
| Talc | 5.50 | 5.50 |
| Glycerin | 0.50 | 0.50 |
| Polyacrylate Microcapsules | 3.00 | 0.00 |
| Containing Feminine Odor | | |
| Control Perfume | | |
| Fragrance | 1.25 | 1.25 |
| Antiperspirant Active | 0.00 | 0.00 |

The cosmetic compositions generally described above can be used in systems for managing incontinence and/or in systems for hair removal from intimate skin and care for the same. In one system for managing incontinence, the system comprises an absorbent article comprising a topsheet, a backsheet, and an absorbent member disposed therebetween; and a cosmetic product comprising an applicator and a cosmetic composition comprising a hardness value of at least 600 gram-force. The absorbent article and/or packaging therefore comprise a first brand identifier. The cosmetic product and/or packaging therefore comprise a second brand identifier. And the first brand identifier and the second brand identifier are the same. A representative, non-limiting list of brand identifiers includes colors, shades, designs, pictures, words, phrases, symbols, shapes, graphics, images, printings, and embossments. In one aspect of the present invention, the packaging of the cosmetic product comprises a color having a hue angle between 90 and 300 degrees. This hue angle generally includes blues and purples, which can connote a cool, soothing product message to consumers.

In a second system for managing incontinence, the system comprises an absorbent article comprising a topsheet, a backsheet, an absorbent member disposed between the topsheet and the backsheet, and a lotion composition that is solid at room temperature but softens at body temperature; and a cosmetic product comprising an applicator and a cosmetic composition comprising a hardness value of at least 600 gram-force. The absorbent article and/or packaging therefore comprise a first brand identifier. The lotion composition and the cosmetic composition are different, in at least one aspect, from one another.

The absorbent articles can take on a variety of different configurations, including, pantiliners, pads (with and without flaps/wings, and with and without cuffs), pants, taped diapers, briefs, etc. Examples of topsheet materials, backsheet materials, absorbent members, and article lotion compositions are described infra.

The absorbent article can comprise any known or otherwise effective topsheet, such as one which is compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. The topsheet, while capable of allowing rapid transfer of fluid through it, also provides for the transfer or migration of the lotion composition onto an external or internal portion of a body of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making absorbent articles such as sanitary napkins, pantiliners, incontinence pads, and the like. When the topsheet comprises a nonwoven fibrous material in the form of a nonwoven web, the nonwoven web may be produced by any known procedure for making nonwoven webs, nonlimiting examples of which include spunbonding, carding, wet-laid, air-laid, meltblown, needle-punching, mechanical entangling, thermo-mechanical entangling, and hydroentangling.

The backsheet can be any known or otherwise effective backsheet material, provided that the backsheet prevents external leakage of exudates absorbed and contained in the absorbent article. Flexible materials suitable for use as the backsheet include, but are not limited to, woven and nonwoven materials, laminated tissue, polymeric films such as thermoplastic films of polyethylene and/or polypropylene, composite materials such as a film-coated nonwoven material, or combinations thereof, as is well known in the art of making absorbent articles, such as sanitary napkins, pantiliners, incontinence pads, and the like

An absorbent member or core is typically positioned between the topsheet and the backsheet. As used herein, the term “absorbent member” refers to a material or combination of materials suitable for absorbing, distributing, and storing aqueous fluids such as urine, blood, menses, and water found in body exudates. The size and shape of the absorbent member can be altered to meet absorbent capacity requirements, and to provide comfort to the wearer/user.

Nonlimiting examples of absorbent member materials include comminuted wood pulp, which is generally referred to as airfelt; creped cellulose wadding; absorbent gelling materials including superabsorbent polymers, such as hydrogel-forming polymeric gelling agents; chemically stiffened, modified, or cross-linked cellulose fibers; meltblown polymers including coform; synthetic fibers including crimped polyester fibers; tissue including tissue wraps and tissue laminates; capillary channel fibers; absorbent foams; absorbent sponges; synthetic staple fibers; peat moss; or any equivalent material; and combinations thereof.

In some embodiments, the absorbent article comprises a lotion composition that is intended to be at least partially transferred to intimate skin when the article is worn. The lotion compositions can be applied in any known manner, in any known pattern, and to typically to the topsheet. The lotion composition can be applied to an entire surface of the absorbent article, such as the topsheet, or to portions thereof, either uniformly or non-uniformly, such as in patches, swirls, stripes, bands, or along the outer edges, or any combination thereof.

The lotion compositions may contain one or more of the following ingredient categories: rheology structurant, surface energy modifier, body treatment agents, carrier, and other optional ingredients.

A “rheology structurant” is a material that helps immobilize lotion ingredients on the topsheet and impede migration through the topsheet into the absorbent core. If the lotion migrates to the absorbent member materials, the absorption properties of thereof may decrease, and more lotion must be applied to the topsheet in order to obtain a beneficial effect. The rheology structurant can also assist in transfer of a portion of the lotion to the skin and delivers target sensory at wearing. Typically, a rheology structurant rapidly solidifies on the topsheet after application of the lotion.

The rheology structurant can be provided in an amount of about 0.1% to 90%, by weight of the lotion composition. In a preferred embodiment, the lotion compositions comprise about 1% to about 50% of a rheology structurant, by weight of the lotion composition. In a more preferred embodiment, lotion compositions comprise about 3% to about 30%, of a rheology structurant, by weight of the lotion composition. A preferred rheology structurant used in a present lotion composition comprises microcrystalline wax.

In addition to a rheology structurant, in certain embodiments, the lotion composition comprises surface energy modifiers, examples of which include POE-4 monolaurate and POE-8 monostearate. The surface energy modifiers can provide a rapid transfer of a relatively mobile portion of the lotion to the skin of the wearer resulting in a film on the skin that resists dissolving in menses and urine, and protects the skin from fouling. The combination of POE-4 monolaurate and POE-8 monostearate with rheology structurant such as microcrystalline wax can also provide a relatively immobile portion of the lotion that remains on the topsheet to facilitate migration of the menses through the topsheet and absorption of the menses by the absorbent core, which helps prevent fouling of the top sheet by the menses for the entire time the absorbent article is used by the wearer.

In addition to the rheology structurant and surface energy modifiers, lotion compositions optionally can further comprise one or more body treatment agents, for example hexamidine, zinc oxide, and niacinamide, which are highly effective in the prevention and treatment of erythema, malodor, and bacterial skin disorders, especially when a lotion composition is administered to the body from application on absorbent articles.

Lotion compositions of the present invention further comprise a carrier. The carrier can be an individual carrier or a blend two or more of carriers, provided that the total carrier concentration is sufficient to provide transfer and/or migration of the rheology structurant, surface energy modifiers, and/or optional body treatment agents onto the body and to promote fluid acquisition into the absorbent article without causing fouling issues. The carrier can be a liquid, solid, or semisolid carrier material, or a mixture of these materials, provided that the resultant carrier forms a homogenous mixture, solution, stable emulsion, or stable dispersion at selected processing temperatures for the resultant carrier system and at processing temperatures for combining the carrier system with the skin treatment agents, surface energy modifiers and rheology structurants in formulating the lotion compositions herein. The preferred carrier system would enable the finished lotion to be semi-solid or solid at ambient, liquid or semi-solid at body temperature and liquid with processible viscosity at 60-90 C.

The lotion compositions of the present invention comprise a carrier in an amount of about 5% to about 95%, preferably about 10% to about 85%, more preferably from about 30% to about 80%, by weight of the lotion composition. Suitable carrier compounds include petroleum-based hydrocarbons having from about 8 to about 32 carbon atoms, fatty alcohols having from about 12 to about 18 carbon atoms, polysiloxane compounds, fatty acid esters, alkyl ethoxylates, lower alcohols having from about 2 to about 6 carbon atoms, low molecular weight glycols and polyols, fatty alcohol ethers having from about 12 to about 22 carbon atoms in their fatty chain, lanolin and its derivatives, ethylene glycol derivatives of C₁₂-C₂₂ fatty acids, glyceride and its derivatives including acetoglycerides and ethoxylated glycerides of C₁₂-C₁₈ fatty acids, and mixtures thereof. Alternatively or in combination with, the carrier also may comprise polysiloxane compounds non-limiting examples include C₁₂-C₂₈ alkyl dimethicones, C₁₂-C₂₈ alkyl methicones, alkylated silicones (hair conditioning agents), silicone waxes, copolymers of silicone (vinyl dimethicone polymers, phenyl vinyl dimethicone polymers, alkylated silicone polymers, polyethylene oxide/silicone copolymers, polyethylene oxide/polypropolene oxide/silicone copolymers, polypropylene oxide/silicone copolymers, polyethylene oxide/alkyl silicone copolymers, polyethylene oxide/propylene oxide/alkyl silicone copolymers, polypropylene oxide/alkyl silicone copolymers), and mixtures thereof.

Lotion compositions of the present invention also can include additional optional components known to persons skilled in the art of lotions for catamenial devices. One such optional ingredient is a fumed silica to adjust lotion viscosity and thickness to a desired level. Additional viscosity and thickening agents are disclosed in U.S. Patent Publication No. 2005/0129651. Other optional ingredients include glycerin, chamomile, skin conditioners, perfumes, deodorants, preservatives, astringents, opacifiers, proteins, urea, lecithin, fats, oils, essential oils, pH control agents, and antibacterials, each in an amount to perform its intended function without adversely affecting the lotion composition, e.g., about 0.001% to about 5% by weight of the lotion composition. Non-limiting exemplary lotion compositions are illustrated in the following Examples 5-10.

### Example 5

| Component | Trade Name | Supplier | Weight % |
|---|---|---|---|
| Microcrystalline | MULTIWAX W-835 | Sonneborn | 17 |
| Wax | | | |
| POE-4 Monolaurate | PEGOSPERSE-200ML | Lonza | 8 |
| POE-8 Monostearate | MYRJ-S8-SO | Croda | 10 |
| Zinc Oxide Premix¹⁾ | Zinc Oxide Premix | Kobo | 7 |
| Fumed Silica | CAB-O-SIL TS-720 | Cabot | 4 |
| Petrolatum | WHITE PROTOPET-1S | Sonneborn | balance |

¹⁾Zinc oxide premix contains 69.8% zinc oxide, by weight, dispersed in the mixture of isononyl isononanoate and polyhydroxstearic acid

### Example 6

| Component | Trade Name | Supplier | Weight % |
|---|---|---|---|
| Microcrystalline | MULTIWAX W-835 | Sonneborn | 14 |
| Wax | | | |
| POE-4 Monolaurate | PEGOSPERSE-200ML | Lonza | 6 |
| POE-8 Monostearate | MYRJ-S8-SO | Croda | 7.5 |
| Lauryl Alcohol | CO-1270 | P&G | 3 |
| Zinc Oxide Premix¹⁾ | Zinc Oxide Premix | Kobo | 7 |
| Fumed Silica | CAB-O-SIL TS-720 | Cabot | 4 |
| Petrolatum | WHITE PROTOPET-1S | Sonneborn | balance |

### Example 7

| Component | Trade Name | Supplier | Weight % |
|---|---|---|---|
| Microcrystalline | MULTIWAX W-835 | Sonneborn | 14 |
| Wax | | | |
| POE-4 Monolaurate | PEGOSPERSE-200ML | Lonza | 4 |
| POE-8 Monostearate | MYRJ-S8-SO | Croda | 4.5 |
| Fumed Silica | CAB-O-SIL TS-720 | Cabot | 4 |
| Petrolatum | WHITE PROTOPET-1S | Sonneborn | balance |

### Example 8

| Component | Trade Name | Supplier | Weight % |
|---|---|---|---|
| Microcrystalline | MULTIWAX W-835 | Sonneborn | 12 |
| Wax | | | |
| POE-4 Monolaurate | PEGOSPERSE-200ML | Lonza | 4 |
| POE-8 Monostearate | MYRJ-S8-SO | Croda | 4.5 |
| Polypropylene | PLURIOL P-4000 | BASF | 4 |
| Glycol - 4000 | | | |
| Zinc Oxide Premix¹⁾ | Zinc Oxide Premix | Kobo | 7 |
| Fumed Silica | CAB-O-SIL TS-720 | Cabot | 4 |
| Petrolatum | WHITE PROTOPET-1S | Sonneborn | balance |

### Example 9

| Component | Trade Name | Supplier | Weight % |
|---|---|---|---|
| Microcrystalline | MULTIWAX W-835 | Sonneborn | 17 |
| Wax | | | |
| C28 Alkyl | Silwax Di5026 | Siltech | 6 |
| Dimethicone | | | |
| POE-4 Monolaurate | PEGOSPERSE-200ML | Lonza | 7.5 |
| POE-8 Monostearate | MYRJ-S8-SO | Croda | 6 |
| Zinc Oxide Premix¹⁾ | Zinc Oxide Premix | Kobo | 7 |
| Fumed Silica | CAB-O-SIL TS-720 | Cabot | 4 |
| Petrolatum | WHITE PROTOPET-1S | Sonneborn | balance |

### Example 10

| Component | Trade Name | Supplier | Weight % |
|---|---|---|---|
| Microcrystalline | MULTIWAX W-835 | Sonneborn | 12 |
| Wax | | | |
| C30 Alkyl | Silwax C41M90 | Clariant | 1 |
| Dimethicone | | | |
| C28 Alkyl | Silwax Di5026 | Siltech | 6 |
| Dimethicone | | | |
| POE-4 Monolaurate | PEGOSPERSE-200ML | Lonza | 10 |
| POE-8 Monostearate | MYRJ-S8-SO | Croda | 15 |
| Zinc Oxide Premix¹⁾ | Zinc Oxide Premix | Kobo | 7 |
| Fumed Silica | CAB-O-SIL TS-720 | Cabot | 4 |
| Petrolatum | WHITE PROTOPET-1S | Sonneborn | balance |

While today's markets contain deodorants and other skin care compositions in the form of sprays, lotions, creams, and washes for use on intimate skin, solid sticks and other solid, rub-on product configurations are believed to be a new product form for treating intimate skin, particularly in the pubis region and neighboring skin regions. Marketing techniques are generally required for introducing new product forms and uses to consumers. And while signage, displays, and product packaging can be effective in communicating information to consumers, these approaches may not be sufficient to inform consumers about systems, regimens, and methods that involve use of two or more different products together. The present disclosure includes several exemplary methods for introducing a new product form for intimate skin care.

In one embodiment, the method comprises the steps of shelving women's shaving products in a store location, and shelving a cosmetic composition in the same store location. A same store location can include the following exemplary scenarios wherein the shaving products and the cosmetic composition are within a same line of sight, in the same aisle, in a common display, and/or positioned within two, three, four, five, six, seven, eight, nine, ten, twelve, or fifteen feet of each other. The cosmetic composition is provided in a stick form (solid at room temperature) within an applicator, wherein a consumer holds the applicator and rubs the stick onto intimate skin for deposition of the some of the composition onto the skin.

The cosmetic composition can contain one or more cosmetic active ingredients directed at managing erythema, pain, inflammation, irritation, itchiness, razor bumps, chaffing, ingrown hairs, and/or dryness. Examples of such ingredients include complexes of PVP and hydrogen peroxides, diclofenac, acetyl salicylic acid, salicylates, ibuprofen, bisabolol, mimosa extract (Mimosa tenuiflora), hyaluronic acid, propylene glycol, glycerines, chondroitin sulfates, plant extracts, bisabolol, panthenol, tocopherol, actives for anti-stinging, anti-irritants, anti-dandruffs, for anti-ageing e.g. retinol, melibiose etc. Other suitable actives are e.g. medicago officinalis, actinidia chinensis, allantoin, Aloe barbadensis, anona cherimolia, anthemis nobilis, arachis hypogaea, arnica montana, avena sativa, beta-carotene, bisabolol, borago, officinalis, butylene glycol, calendula officinalis, camellia sinensis, camphor, candida bombicola, capryloyl glycinee, carica papaya, centaurea cyanus, cetylpyridinium chloride, Chamomilla recutita, chenopodium quinoa, chinchona succirubra, chondrus crispus, Citrus aurantium dulcis, Citrus grandis, Citrus limonum, Coco-nucifera, Coffea arabica, Crataegus monogina, Cucumis melo, dichlorophenyl imidazoldioxolan, enteromorpha compressa, equisetum arvense, ethoxydiglycol, ethyl panthenol, farnesol, ferulic acid, fragaria chiloensis, gentiana lutea, Ginkgo biloba, glycerine, glyceryl laurate, glycyrrhiza glabra, hamamelis virginiana, heliotropine, hydrogenated palm glycerides, Citratess, hydrolyzed castor oil, hydrolyzed wheat protein, hypericum perforatum, Iris florentina, juniperus communis, lactis proteinum, lactose, lawsonia inermis, linalool, linum usitatissimum, lysine, magnesium aspartate, magnifera indica, malva sylvestris, mannitol, mel, melaleuca alternifolia, mentha piperita, menthol, menthyl lactate, Mimosa tenuiflora, Nymphaeaalba, olaflur, oryza sativa, panthenol, paraffinum liquidum, PEG-20M, PEG-26 Jojoba acid, PEG-26 Jojoba alcohol, PEG-35 castor oil, PEG-40 hydrogenated castor oil, PEG-60 hydrogenated castor oil, PEG-8 caprylic/capric acid, persea gratissima, petrolatum, potassium aspartate, potassium sorbate, propylene glycol, prunus amygdalus dulcis, prunus armeniaca, prunus persica, retinyl palmitate, ricinus communis, rosa canina, rosmarinus officinalis, rubus idaeus, salicylic acid, sambucus nigra, sarcosine, serenoa serrulata, simmondsia chinensis, sodium carboxymethyl betaglucan, sodium cocoyl amino acids, sodium hyaluronate, sodium palmitoyl proline, stearoxytrimethylsilane, stearyl alcohol, sulfurized TEA-ricinoleate, talc, Thymus vulgaris, tilia cordata, tocopherol, tocopheryl acetate, trideceth-9, triticum vulgare, tyrosine, andecylenoyl glycinee, urea, vaccinium myrtillus, valine, zinc oxides, zinc sulfates, and combinations thereof.

Other methods of the present invention comprise shelving the cosmetic composition in the same store location as other intimate hair management products such as waxing products and depilatories, for example. And still other methods comprise shelving the cosmetic composition in the same location as personal lubricants and other products (e.g., feminine sprays, washes, and lotions) typically used on intimate skin. In yet another embodiment, the method comprises shelving the cosmetic composition in the same store location as undergarments and swim suits that may contain a cut or configuration that would expose pubic hair if such hair is not removed.

Methods of the present invention can similarly comprise the steps of shelving absorbent articles in a store location, and shelving a cosmetic composition in the same store location. The absorbent articles can include incontinence products, menstrual products, and everyday products such as pantiliners for managing odor and other low volume bodily fluid discharges.

The present disclosure also includes several exemplary methods for informing retailers and consumers about a new product form for intimate skin care. In one embodiment, the method comprises the steps of providing a cosmetic product that comprises an applicator and a cosmetic composition, and communicating consumer research data to a retailer to support a display location of the cosmetic product that is proximate women's shaving products in the retailer's store. The type of consumer research data communicated to the retailer is not limited. It can include, for example, concept test data, focus group data, mock shelf/store data, qualitative consumer data, quantitative consumer data, etc. The data can include numbers, charts, and graphical representations. The research data can include consumer knowledge and behavior, including, for example, product awareness, product use, and product purchase intent. The data may support the display location based on positive monadic retail data. The data may also or alternatively support the display location based on higher positive retail data in comparison to displaying the cosmetic product in a different location.

Besides in-store merchandising, the present invention also contemplates methods involving electronic devices (e.g., computers, tablets, and smart phones). Consumers searching for and/or purchasing intimate skin area hair removal products, adult incontinence products, swimsuits, undergarments, and the like, can be served or otherwise communicated information (e.g., via targeted advertising) regarding the cosmetic products described herein. The information can include, but is not limited to, introduction to a new product form and use, coupons, locations for purchasing the cosmetic product, tips on usage, and benefits associated with usage.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A system for managing incontinence, the system comprising:
a. an absorbent article comprising a topsheet, a backsheet, and an absorbent member disposed between the topsheet and the backsheet; and
b. a cosmetic product comprising an applicator and a cosmetic composition disposed within the applicator that is solid at room temperature;
c. wherein the absorbent article comprises a lotion composition;
d. wherein the lotion composition is different than the solid cosmetic composition.

2. The system of claim 1, wherein the absorbent article and the cosmetic product are packaged together.

3. The system of claim 1, wherein the absorbent article and the cosmetic product are displayed in a store proximate one another.

4. The system of claim 1, wherein the absorbent article and the cosmetic product are displayed in a store within 10 feet of one another.

5. The system of claim 1, wherein the cosmetic composition is anhydrous.

6. The system of claim 1, wherein the cosmetic composition comprises less than 1% of a glycol.

7. A system for managing incontinence, the system comprising:
a. an absorbent article comprising a topsheet, a backsheet, and an absorbent member disposed between the topsheet and the backsheet, the absorbent article and/or packaging therefore comprising a first brand identifier; and
b. a cosmetic product comprising an applicator and a cosmetic composition disposed within the applicator, the cosmetic composition comprising a hardness value of at least 600 gram-force, and a second brand identifier associated with the cosmetic product and/or packaging therefore;
c. wherein the first brand identifier and the second brand identifier are the same.

8. The system of claim 7, wherein the absorbent article and the cosmetic product are packaged together.

9. The system of claim 7, wherein the absorbent article and the cosmetic product are displayed in a store proximate one another.

10. The system of claim 7, wherein the absorbent article and the cosmetic product are displayed in a store within 10 feet of one another.

11. The system of claim 7, wherein the cosmetic composition is anhydrous.

12. A system for shaving and caring for intimate skin, the system comprising:
a. a razor for shaving hair in the intimate skin area; and
b. a cosmetic composition for applying to the intimate skin area before and/or after shaving hair from the same, the cosmetic composition comprising a hardness of at least 600 gram-force requiring it to be rubbed onto the intimate skin area for deposition.

13. The system of claim 12, wherein the cosmetic composition further comprises one or more ingredients directed at managing erythema, pain, itchiness, razor bumps, chaffing, ingrown hairs, and/or dryness.

14. The system of claim 12, wherein the cosmetic composition further comprises a Vitamin B₅ derivative.

15. The system of claim 14, wherein the Vitamin B₅ derivative is panthenyl triacetate.

16. The system of claim 12, wherein the razor and the cosmetic composition are packaged together.

17. The system of claim 12, wherein the razor and the cosmetic composition are displayed in a store proximate one another.

18. The system of claim 12, wherein the razor and the cosmetic composition are displayed in a store within 10 feet of one another.

19. The system of claim 12, wherein the cosmetic composition is anhydrous.

20. The system of claim 12, wherein the cosmetic composition comprises less than 1% of a glycol.
